(19) United States
(12) Patent Application Publication
(10) Pub. No.: US 2006/0276797 A1
(43) Pub. Date: Dec. 7, 2006

(54) Expandable reaming device

(76) Inventor: Gary Botimer, Nampa, ID (US)

Correspondence Address: PEDERSEN & COMPANY, PLLC, P.O. BOX 2666, BOISE, ID, 83701, US

(21) Appl. No.: 11/137,123

(22) Filed: May 24, 2005

(51) Int. Cl. A61B 17/00

(52) U.S. Cl. 606/81

(57) ABSTRACT

An expanding reamer for reaming or cutting a concave surface, for example, for reaming an acetabulum in preparation for implanting a prosthetic component, such as an acetabular cup or socket, during a hip arthroplasty. The reamer includes a rotating shaft cooperating with a surgical drill or other power source at one end and rotating a reamer head at the other end, and a system adapted to expand one or more blades on the reamer head. In a preferred version, the reamer head comprises a plurality of generally circular, preferably substantially flat and parallel blades, the outer blades of which are radially expandable as segments of a cutting sphere to enlarge the effective diameter of the reamer head. A transverse blade may guide expansion of the blades to move upwards as well as outward to maintain a nearly perfect cutting sphere across a range of diameters. Upon rotation of the reamer head, the blades form a portion of an effective cutting sphere that is preferably greater-than-180-degrees in order to allow greater flexibility in placement of the shaft of the reamer relative to the surface being reamed, for example, relative to the center of axis of the acetabulum.

### BACKGROUND OF THE INVENTION

1. Field of the Invention

The present invention relates generally to a reaming device and, more particularly, to an expandable reaming device that may be used for reaming an acetabulum in preparation for implanting a prosthetic component, such as an acetabular cup or socket, during a hip arthroplasty.

2. Related Art

The hip joint is a ball-and-socket joint formed by the articulation of the rounded, convex surface of the head of the femur with the cuplike acetabulum on the pelvis. In a healthy hip joint, the head of the femur and the acetabulum are lined by surface cartilage; the entire joint is surrounded by a capsule which has a thin lining of synovial cells that produce a thin layer of lubrication film, called synovial fluid. The synovial fluid together with the cartilage acts as a shock absorber and allows the joint to move. If the surface cartilage is badly damaged, or if the joint surfaces are not aligned properly, then the cartilage will wear out, and as a result, the bone under the cartilage layer is exposed. The exposed bone starts to rub against each other and the process of osteoarthritis is established.

Osteoarthritis is the result of mechanical wear and tear on a joint, in this case the hip joint. The main indication is a loss of surface cartilage due to the bone rubbing on bone. The formation of bone spurs, called osteophytes and cysts around the joint is another indication of osteoarthritis. The body tries to relieve the pain from the rubbing of the bone by increasing the amount of fluid in the joint. In an arthritic hip, the cartilage lining is thinner than normal or completely absent; the capsule of the arthritic hip is swollen; the joint space is narrowed and irregular in outline; and/or excessive osteophytes can build up around the edges of the joint. The combination of these factors cause pain and will eventually result in the loss of motion of the hip.

Hip arthroplasty is a surgery performed to replace all or part of a joint deteriorated from osteoarthritis with an artificial device to restore joint movement. There are different types of hip arthroplasty. If a hemi-arthroplasty is performed, the femoral head or the acetabulum is replaced with a prosthetic. In a total hip arthroplasty, both the femoral head and the acetabulum are replaced with a prosthetic device. Hip arthroplasty involves reforming the patient's natural acetabulum, so that a proper bearing surface of the ball of a femur is established in order to support the normal motion of the leg. The acetabulum needs to be shaped so that it can receive a metallic or plastic artificial socket. To ensure a proper fit of the artificial socket, osteophytes and other deteriorated and diseased bone are removed from within and around the acetabulum using a bone chisel, until healthy bone becomes visible. Typically, a reamer is used to reshape the acetabulum; however, reamer heads of increasingly larger size are required as bone is cut away and the socket is enlarged. Each time a larger reamer head is needed, the reaming system must be removed from the patient's acetabulum, the reamer head is removed from the drive shaft of the surgical drill, and the next larger reamer head is attached. This sequence may be repeated several times until the acetabulum is completely prepared to receive an acetabular prosthetic implant. The process of replacing reamer heads multiple times during a surgery is time consuming, inefficient, inconvenient, and may also lead to surgical errors in that the angle of acetabular penetration cannot be accurately preserved during each reamer head substitution.

Additionally, standard hip arthroplasty is typically performed using a posterolateral or anterolateral approach, with an incision of 25-30 cm in length (see FIG. 1A). The approach provides complete and continuous observation of the hip; however, this exposure comes at the expense of trauma to the muscle and tendons and considerable postoperative pain, requiring inpatient stay and delay of postoperative physical therapy. Recently, minimally invasive (MIS) hip arthroplasty has been used as an alternative. MIS hip arthroplasty approaches include single-incision and 2-incision techniques, each measuring about 10 cm in length (see FIG. 1B). The decrease in muscle and tendon trauma is achieved at the expense of the complete and continuous observation of the hip. Additionally, the small incision makes it more difficult to place the acetabular reamer in direct alignment with the axis of the acetabulum. With the larger incision, it was less likely that the reamer would be off axis; however, if the smaller incision is not exactly aligned with the acetabulum (see FIG. 2A), the reamer will be off axis with the axis of the acetabulum. If the reamer is off axis and the head of the reamer has hemispherical or less cutting capability (“180 degrees or less head”), it will be unable to cut a perfect hemisphere in the acetabular space (see FIG. 2B). A portion P of the acetabular space will be improperly reamed, or, more likely, not reamed at all. Therefore, the inventor believes that there is still a need for an acetabular reamer that is expandable to eliminate the need for multiple reamers and a reamer head that is greater than 180 degrees to allow the surgeon to cut a perfect hemisphere even when the reamer is off axis.

Issued patents relating to expandable acetabular reaming devices are reviewed hereinafter.

Fishbein (U.S. Pat. No. 3,702,611) discloses an expanding reamer including a head with a convex end adapted to seat in a previously prepared concavity in the central part of the acetabulum; the head pivotally mounts a set of radially expansive blades and is telescopically mounted on the end of a rotary drive shaft.

Temeles (U.S. Pat. No. 6,283,971) discloses an expandable acetabular reaming system having a plurality of blades which project or retract through a reamer head according to a desired reamer head size. The degree projection or retraction of the reaming blades is manually controlled by user actuation of an air bladder.

### SUMMARY OF THE INVENTION

The present invention relates to an expandable reaming device for reaming, cutting, or drilling, which has one or more moveable blades for increasing the effective diameter of the reamer head. The expandable device may be adapted for reaming an acetabulum in preparation for implanting a prosthetic component, such as an acetabular cup or socket, during a hip arthroplasty. The preferred reaming device comprises blades or blade portions that, individually or together, provide a greater-than-180-degree cutting edge(s), so that, upon rotation of the reamer head, the device may ream a hemisphere in a surface even if the rotational axis of the device is not parallel to the axis of the concave surface being reamed/cut. During a hip arthroplasty, this offers greater flexibility in placement of the shaft of the reaming device relative to the center of axis of the acetabulum.

The preferred reaming device comprises a drill bit on a rotating shaft for cooperating with a surgical drill, a plurality of blades connected directly or indirectly to the rotating shaft, and a gearing system to expand the moveable blade or blades radially. Preferably, the moveable, “expanding blade(s)” comprise two parallel blades that remain parallel to each other and to the rotational axis throughout expansion, the blades each being greater than 180 degrees in circumference and each generally forming a segment of a sphere. As the expanding blades move outward, the effective diameter of the reaming head increases and the reaming head may ream/cut increasingly larger-diameter partial spheres. The expanding blades are preferably raised as they are moved outward, to maintain the effective reaming/cutting shape of the reaming head very close to a perfect partial sphere.

In the preferred embodiment, the two expanding blades are located on either side of, and parallel to, a central blade passing through the axis of rotation of the device. A transverse blade is preferably positioned perpendicular to the expanding blades and the central blade, and centered so that it also passes through the axis of rotation of the device. The central blade and/or the transverse blade may also be used for reaming/cutting, especially reaming/cutting of the bottom region of the concave surface being formed, and especially after the moveable blades have been expanded outward. The preferred expanding blades move out along the transverse blade, guided by ramps in or on the transverse blade that raise the blades at the same time they are expanding.

### BRIEF DESCRIPTION OF THE DRAWINGS

In the following drawings, gear teeth and threads are not drawn, but are understood when parts are described by the terms “gear”, “teeth”, “threads,” “threaded,” “toothed surfaces,” or “threaded surfaces.”

FIG. 1A is a front/anterior view of the standard incision made during a total hip arthroplasty.

FIG. 1B is a front/anterior view of the new incision made during a minimally invasive total hip arthroplasty.

FIG. 2A is a schematic illustrating the standard acetabular reamer when the reamer is aligned with the axis of the acetabulum.

FIG. 2B is a schematic illustrating the standard acetabular reamer when the reamer is not aligned with the axis of the acetabulum.

FIG. 3 is a perspective view of one embodiment of the invented acetabular reamer.

FIG. 4A is a front view of the embodiment shown in FIG. 3, with the rear view being the same due to the preferred symmetry of the device.

FIG. 4B is a right side view of the embodiment shown in FIGS. 3-4A, with the left view being the same due to the preferred symmetry of the device.

FIG. 4C is a top view of the embodiment of FIG. 4A, used to show the direction of cross-sectional views for FIGS. 5A and 5B.

FIG. 5A is a right side cross-sectional view of the embodiment shown in FIGS. 3-4B, viewed along the line 5A-5A in FIGS. 4A and 4C.

FIG. 5B is a rear cross-sectional view of the embodiment shown in FIGS. 3-5A, viewed along the line 5B-5B in FIGS. 4B and 4C.

FIG. 6 is an exploded version of the right side cross-sectional view of FIG. 5A.

FIG. 7 is a partial exploded view of the embodiment shown in FIGS. 3-6B, featuring the planetary transmission system used for adjusting the moveable blades.

FIG. 8A is a bottom view of the bottom plate of the embodiment shown in FIGS. 3-7.

FIG. 8B is a side view of the bottom plate of the embodiment shown in FIGS. 3-8A.

FIG. 8C is a top view of the bottom plate of the embodiment shown in FIGS. 3-8B.

FIG. 9 is a side view of the central rod of the embodiment shown in FIGS. 3-8C.

FIG. 10A is a side view of the head of the reamer of the embodiment shown in FIGS. 3-9.

FIG. 10B is a cross-sectional view of the head of the reamer of FIGS. 3-9, viewed along the line 10B-10B in FIG. 10A.

FIG. 10C is a cross-sectional view of the head of the reamer of FIGS. 3-9, viewed along the line 10C-10C in FIG. 10A.

FIG. 10D is a cross-sectional top view of the head of the reamer of FIGS. 3-9, viewed along the line 10D-10D in FIG. 10A.

FIG. 11A is an exploded view of the head of the reamer of the embodiment shown in FIGS. 3-10D.

FIG. 11B is a front exploded view of the head of FIG. 11A.

FIG. 12A is a front view of the worm of the embodiment shown in FIGS. 3-11B, wherein the worm is shown without its teeth and threads.

FIG. 12B is an end view of a worm gear of the embodiment shown in FIGS. 3-12A, wherein the worm gear is shown without its teeth.

FIG. 13A is a front view of the central blade and guide blade combination of the embodiment shown in FIGS. 3-13A.

FIG. 13B is a cross-sectional view of the central blade of the embodiment shown in FIGS. 3-12B, viewed along the line 13B-13B in FIG. 13A.

FIG. 14 is a top view of the central blade and guide blade of the FIG. 13A.

FIG. 15A is a first side view of the gear plate of the embodiment shown in FIGS. 3-14.

FIG. 15B is a top view of the gear plate of FIG. 15A.

FIG. 15C is a second (opposing) side view of the gear plate of FIGS. 15A and B.

FIG. 15D is an end view of the gear plate of FIGS. 15A-C.

FIGS. 16A-16E are front views of the reamer head of the embodiment shown in FIGS. 3-15D, as the cutting blades are expanding.

FIG. 17A is a schematic top cross-sectional view of a fully-contracted reamer head, showing in dashed lines the effective cutting diameter of the head.

FIG. 17B is a schematic top cross-sectional view of the reamer head of FIG. 17A (same size blades) in a fully-expanded condition, again showing in dashed lines the effective cutting diameter of the head.

FIG. 18A is a schematic illustrating a generalized embodiment of the invented acetabular reamer when the reamer is aligned with the axis of the acetabulum.

FIG. 18B is a schematic illustrating the generalized embodiment of the invented acetabular reamer when the reamer is not aligned with the axis of the acetabulum

### DESCRIPTION OF THE PREFERRED EMBODIMENTS

Referring to the figures, there is shown one, but not the only embodiment of the invented expandable reaming device. While the preferred embodiment is especially-well adapted for reaming an acetabulum in hip arthroplasty, other embodiments may be useful for other reaming, cutting, and drilling applications, both in the human body, animals, and/or other applications. Therefore, the terms “reaming,” “cutting,” and “reaming device” are not intended to limit the invented device to a particular medical procedure.

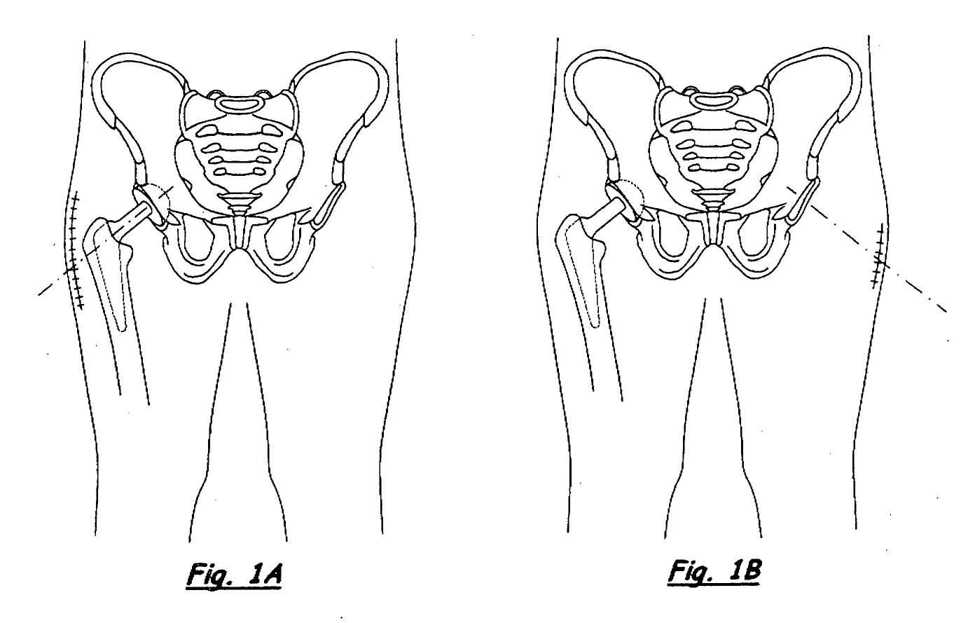

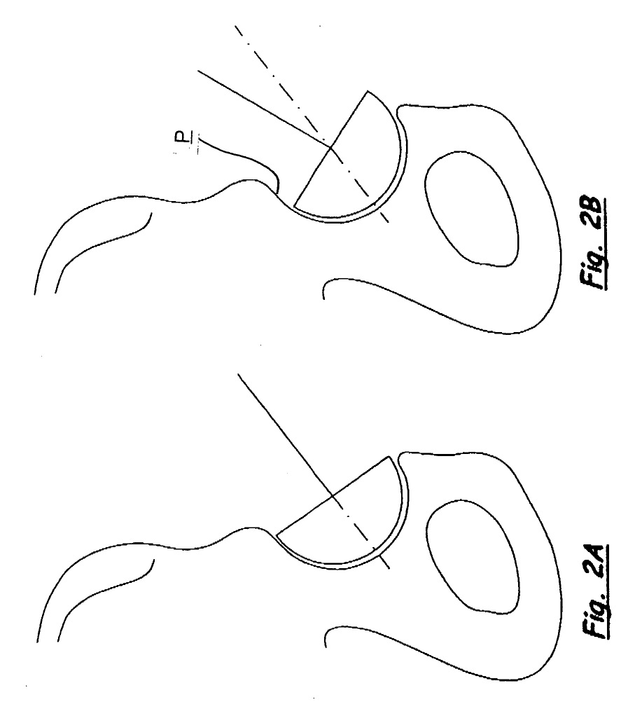

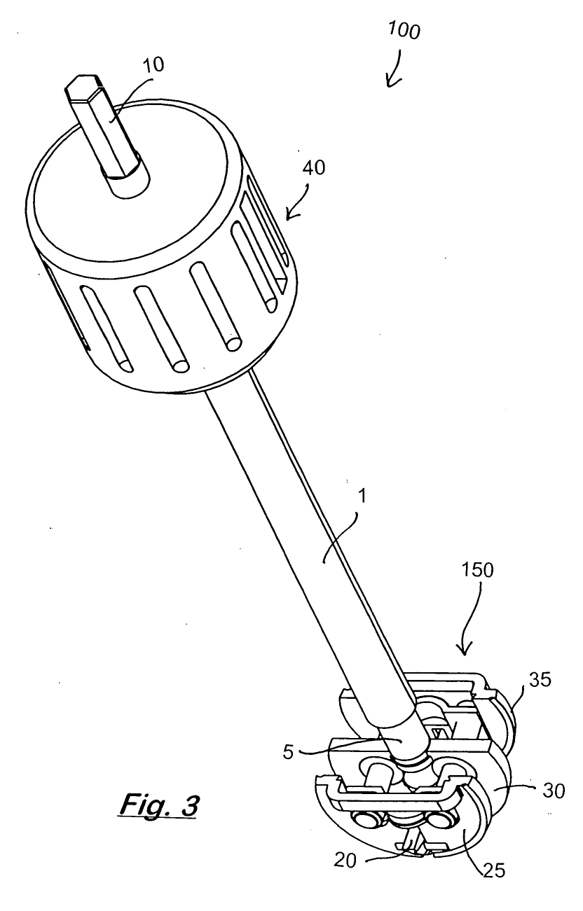

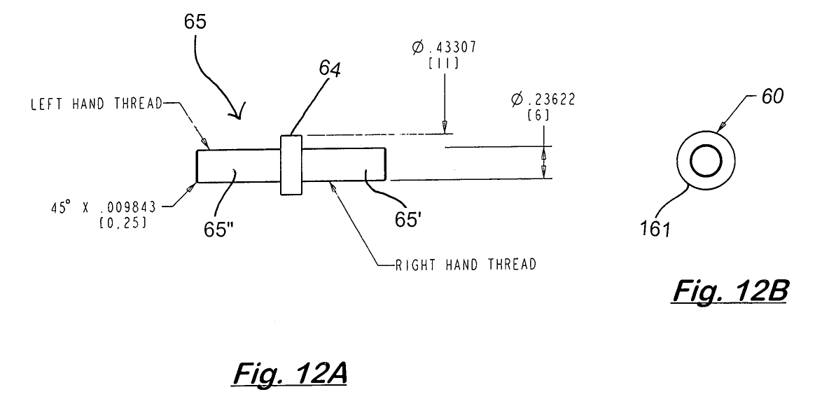

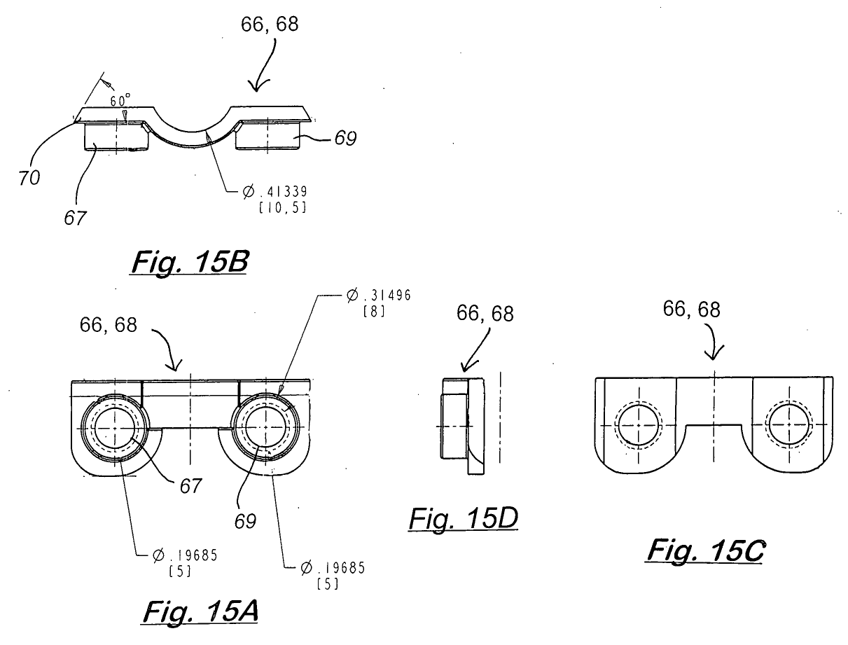

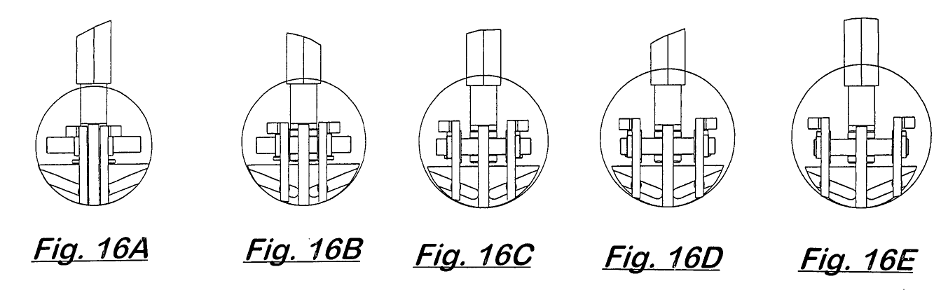

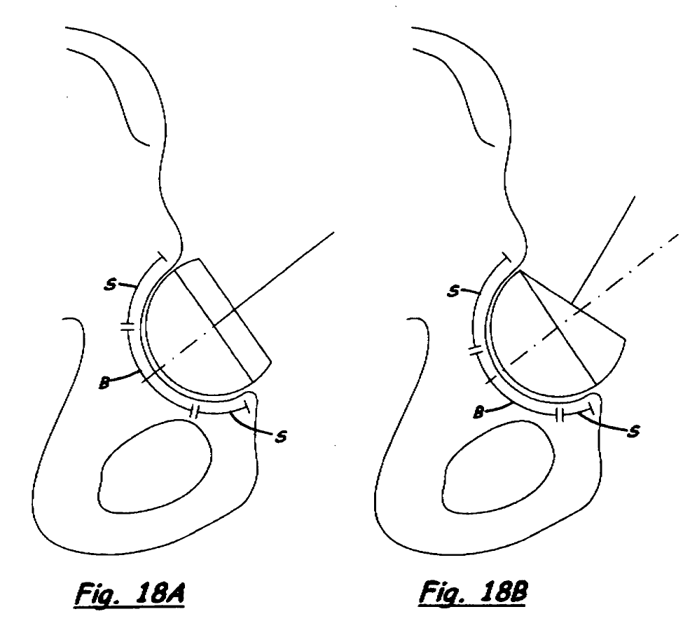

FIGS. 1A and B, and 2A and B illustrate prior art surgical techniques for hip arthroplasty. FIGS. 3-15C illustrate the preferred reaming device and pieces-parts thereof. FIGS. 16A-E, and 17A and B illustrate the preferred expansion structure and methods, and FIGS. 18A and B schematically illustrate the invented reaming device in use reaming the acetabulum.

In general, the preferred reaming device may be described as an elongated tool having a reamer head at one end and a bit or other connection or handle for receiving power at the opposing second end. The reaming device has expandable blades that may be actuated from at or near the second end of the device so that the surgeon may do so while the reamer head is inside the patient. The expansion actuation may be done by a gear system that transmits rotation of a knob or other control member near the second end of the device to rotation of an elongated member that is preferably coaxial with the central axis of the device and that extends down to the reamer head. In the preferred embodiment, said elongated member operates a worm gear assembly in or near the reamer head that transmits rotation of the elongated member to rotation of at least one worm at 90 degrees to the central axis of the device. This rotation, at 90 degrees to the central axis of the device, can be used to move the expandable blades in and out in a direction transverse to the central axis.

Preferably, multiple cutting blades are provided, wherein at least one has a cutting edge extending greater than 180 degrees, or a group of cutting edges that together total greater than 180 degrees. Said cutting edge is, or said group of cutting edges totals, preferably 200-270 degrees, or more preferably 220-250 degrees. Alternatively, a combination of two or more blades may have cutting edges that, when the reamer head is rotated 360 degrees, together are capable of cutting greater than a hemisphere, preferably 200-270 degrees, or more preferably 220-250 degrees. This way, no matter what the orientation of the reamer head in the acetabulum, the reamer head can cut approximately a hemisphere to receive the hemispherical prosthetic socket.

The expansion of the reamer head is done with preferred structure and methods that provide extremely accurate reaming of various hemispherical diameters. At least one of the preferred moveable cutting blades serves as a segment of the “cutting sphere” (more precisely, a segment of a sphere with a spherical cap removed). When the segment is moved outward, transversely to the axis of rotation of the reaming head, that same segment, in effect, becomes a segment of a larger cutting sphere. Therefore, by moving at least one “cutting segment” outward, the effective spherical diameter of the rotating reamer head increases so that the diameter of the reamed surface also increases.

Preferably, two of these blades acting as “cutting segments” are provided, parallel to each other and moveable outward on opposite sides of the head. The preferred segments each have a leading cutting edge that is greater than 180 degrees on a single radius (being a portion of a circumference). This provides a set of two greater-than-180-degree cutting edges, following the same rotational path, but on opposite sides of the head, for providing a balanced head and for increasing the total length of cutting edge. The circular edge of each of the segments is mainly for reaming the “sides” S of the acetabulum, especially as the segments are moved out from the central axis of the reamer head, because, in effect, they rotate around the central axis of the head a distance from the axis.

The circular edge of an additional blade extending parallel to and through the central axis is used for cutting the “bottom” B of the acetabulum, that is, the curved bottom surface of the acetabulum (starting from the center axis of the head and extending out a distance generally equal to said distance of the cutting segments from the axis). In the preferred reamer head, two blades are provided that extend parallel to and through the axis of rotation and that are preferably perpendicular to each other. One or both of these blades, or portions of one or both of these blades may be sharpened or otherwise shaped for cutting/reaming the bottom B. One or both of these blades should have edges or portions or their edges that together or individually, upon a revolution of the reamer head, ream the bottom B in the area from the central axis of the tool to the radial location of the cutting segments. One or both may have a portion that, instead of cutting, mainly moves bone material out of the way after it has been cut from the acetabulum by the other blades. See FIGS. 18A and 18B, illustrating the “sides” S portions and “bottom” B portion of the acetabulum, wherein these portions change depending on the orientation of the device in the acetabulum. Also, these portions will change as the reamer head is expanded (not shown in FIGS. 18A and B). The portion of the acetabulum being reamed by the blade(s) extending through the central axis will increase, while the portion being reamed by the cutting segments will decrease.

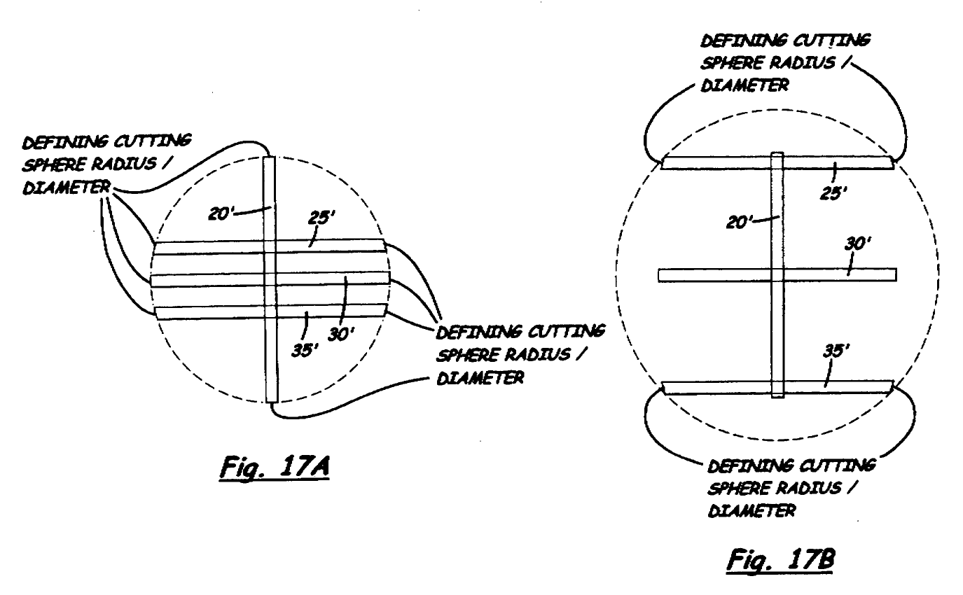

The cutting segment structure and method of expanding the reamer head may be better understood by viewing FIGS. 16A-E, which show progressive stages of expansion, and FIGS. 17A and B, in which the expansion is exaggerated, compared to that normally desired in a surgical reaming device, for the sake of clarity. In FIGS. 17A and B, the moveable blades are called-out as 25′ and 35′, the central blade is called-out as 30′, and the transverse blade is called-out as 20′.

Referring specifically to FIGS. 3-15C:

As shown in FIG. 3, the preferred embodiment of the invented expandable reamer 100 comprises a rotating shaft 5; a drill bit 10 on one end of the rotating shaft 5 for cooperating with a surgical drill; an expansion control rod 15 inside said rotating shaft 5; a reamer head 150 comprising three cutting blades 25, 30, 35 and a transverse guide blade 20 operationally connected to the rotating shaft 5; a knob 40 for actuating a gearing system and the expansion control rod 15 to expand radially at least one and preferably two of the cutting blades to be the “cutting segments” described above. Preferably, a portion of the rotating shaft 5, with the expansion control rod 15 inside the shaft, is contained within a handle sleeve 1.

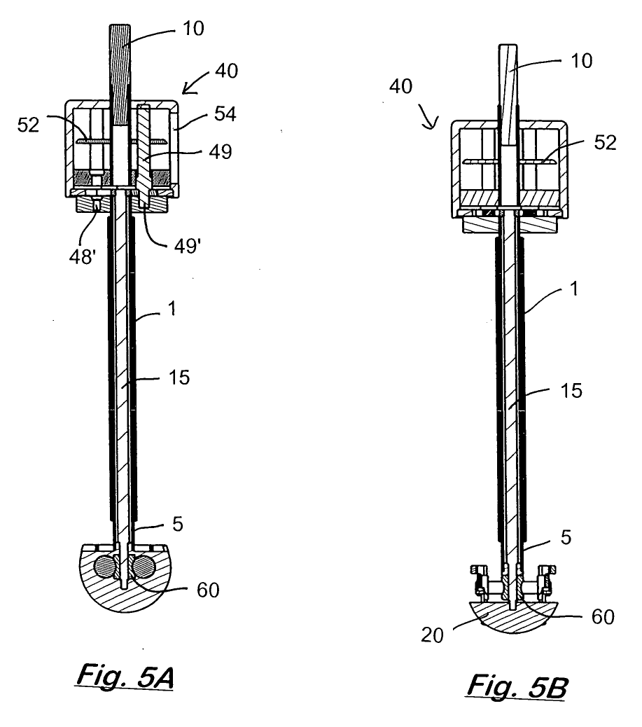

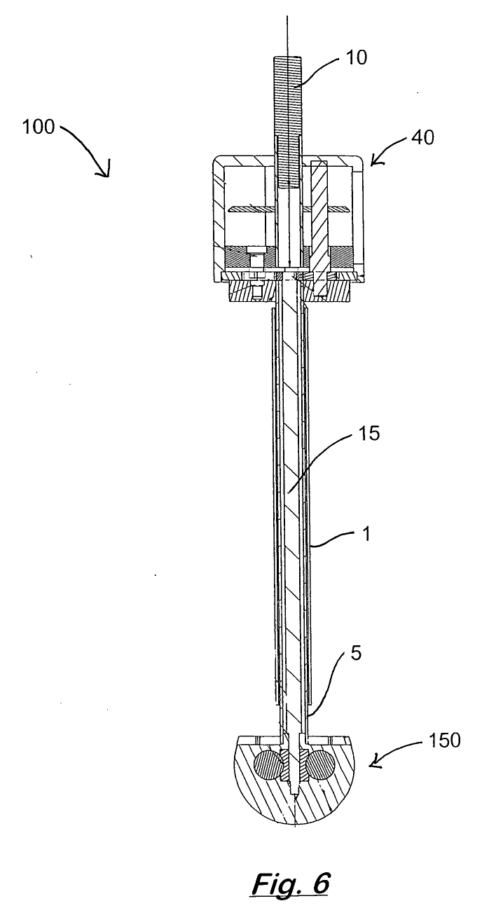

As shown in FIGS. 5A, 5B and 6 to best advantage, the drill bit 10 is rigidly connected to the rotating shaft 5, and the rotating shaft is preferably rigidly connected to the central cutting blade 30, wherein “connected” may imply a direct connection, or an indirect connection including intermediate or intervening connectors. The guide blade 20 is preferably fixed to and perpendicular to the central blade 30, and the expandable blades 25, 35 ride on worms mounted in the central blade 30 and are guided by sloped channels 22, 24 in the guide blade 20. Therefore, rotating the bit 10 rotates the shaft 5, which rotates the entire reamer head 150.

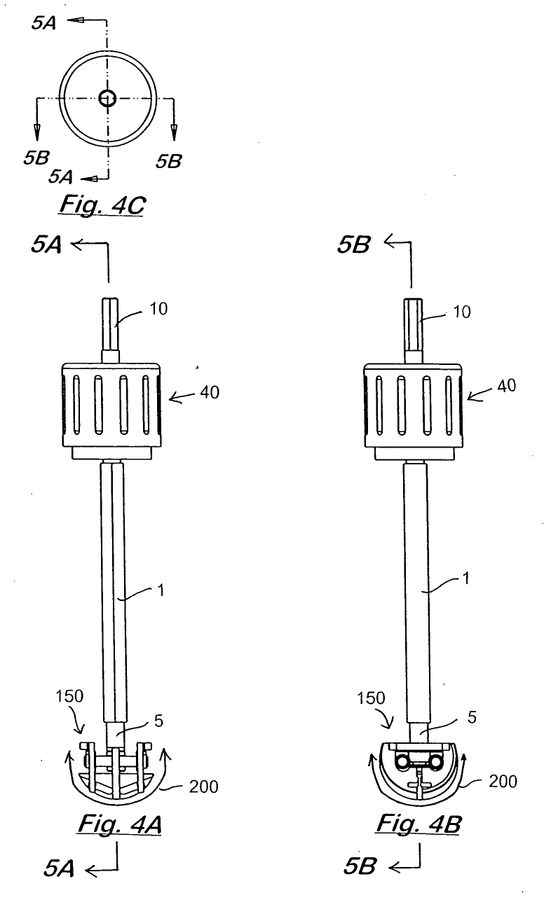

As illustrated to best advantage in FIGS. 4A and 4B, each of the three parallel cutting blades 25, 30, 35 has an outer circumference curving on its respective single radius and is greater than 180 degrees (preferably 200-270 degrees, and more preferably 220-250 degrees), so that, when the reamer head is rotated, the cutting blades 25, 30, 35 are capable of cutting/reaming a portion of a sphere 200 that is 180 degrees or greater than 180 degrees. More precisely, in the preferred application, the sphere portion 200 is capable of cutting/reaming a hemisphere in the acetabulum even when off-axis relative to the acetabulum.

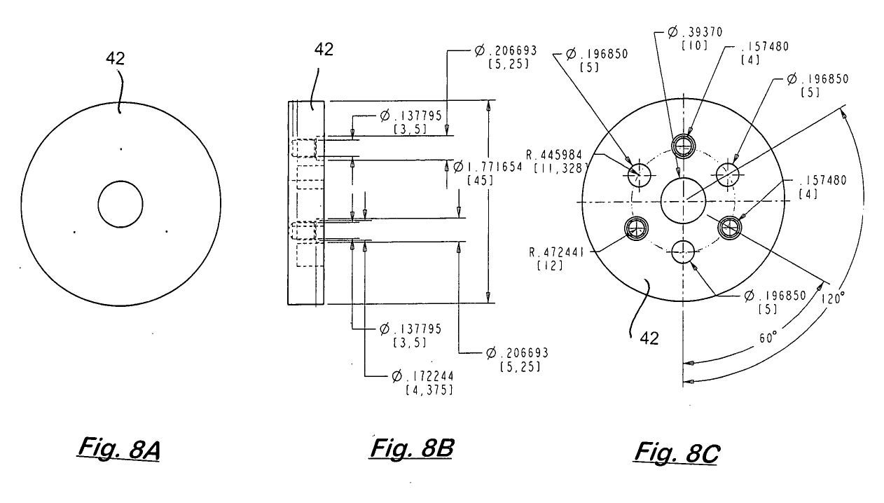

Knob 40 is rotated relative to the rotating shaft 5, in order to actuate the gearing system that expands cutting blades 25, 35. The knob 40 does not rotate with the shaft 5 as the shaft is turned by the drill, and the knob 40 is typically operated only when the user has stopped rotation of the shaft 5 and the reamer head 150. The knob 40 is preferably manually operated and houses or connects to a bottom plate 42, a top plate 45, a planetary gear system 41, a bottom plate 42 (see FIGS. 8A-8C), and an indicator disk 52.

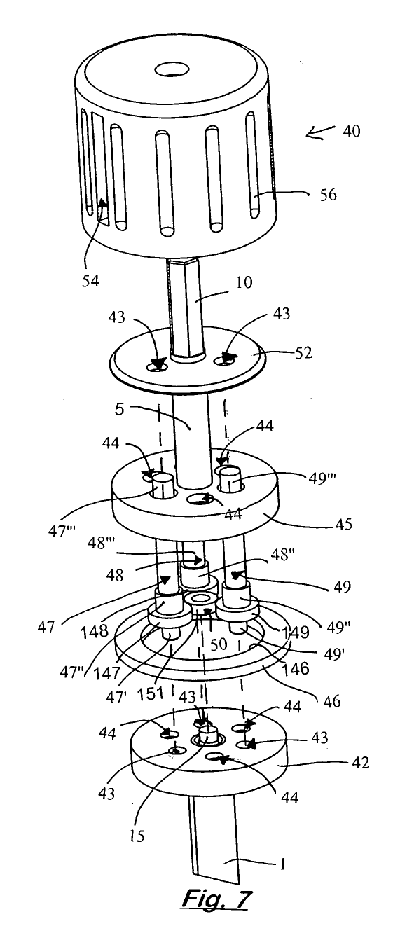

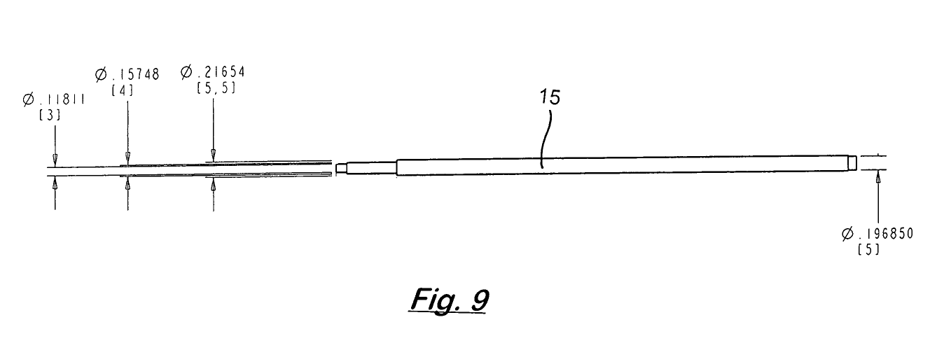

As shown in FIG. 7, the planetary gear system 41 comprises a system of spur gears in which the toothed inner surface 146 of an outer gear ring 46 turns three inner planet gears 47, 48, and 49, which in turn drive a central sun gear 50. The outer perimeter of the gear ring 46 is rigidly attached to the bottom of the knob 40, and the sun gear 50 is rigidly connected to the expansion control rod 15 (see FIGS. 7 and 9). The three planet gears 47, 48, and 49 each comprise a bottom portion 47′, 48′, and 49′, toothed portion 147, 148, and 149 for meshing with the toothed inner surface 146 of the ring 46 and the toothed outer surface 150 of the sun gear, a sleeve portion 47″, 48″, and 49″, and a top portion 47′″, 48′″, and 49′″. In the preferred embodiment, the gear ring 46, the toothed portion 147, 148, and 149 and the sleeve portion 47″, 48″, and 49″, and the sun gear 50 are contained between the bottom plate 42 and top plate 45.

Preferably, the bottom plate 42 and top plate 45 each contain six apertures: three apertures 43 for the planet gears 47, 48, 49 and three apertures 44 for screws to hold the bottom plate 42 and top plate 45 together. The bottom portions 47′, 48′, and 49′ of the planet gears insert into the apertures 43 in the bottom plate 42, the top portions 47′″, 48′″, and 49′″ extend up through the apertures in the top plate 45 and through planet gear apertures 43 in the indicator disk 52. The two plates 42 and 45 are held together by screws (not shown) which insert into the screw apertures in the top and bottom plates 45, 42. The two plates 42 and 45 are separated by the sleeve portions 47″, 48″, and 49″ on the planet gears to give the gear ring 46 and planet gears 47, 48, 49 room to rotate.

In the preferred embodiment, one of the planet gears 49 is threaded on its top portion 49′″. The indicator disk 52 threadably engages the top portion 49′″ while the other two planet gears 47 and 48 merely pass through the apertures 43 in the indicator disk without engaging the disk 52. The knob 40 comprises one or more viewing windows 54 for viewing the indicator disc 52.

As the knob 40 is turned, the gear ring 46 also turns, in turn rotating the planet gears 47, 48, and 49, which rotate the sun gear 50, which rotates the rod 15, in turn expanding the two outer cutting blades 25 and 35 via a worm gear system as will be discussed below. As the planet gears 47, 48, and 49 are rotating, the preferred indicator disk 52 rides up and down on the threaded planet gear top end 49′″, with how far it moves indicating how far the blades 25, 35 have expanded. There may be indicia on the knob 40 surface outside the viewing window(s) 54 to allow the surgeon to know exactly how far out the blades 25 and 35 have moved. Further, the indicating disk may be a color such as red to aid in seeing the indicator disk 52 through the viewing window 54; other colors besides red may be used as long as they are easily visible. Additionally, the knob 40 may be fitted with traction bumps 56 to aid in gripping and turning the knob 40.

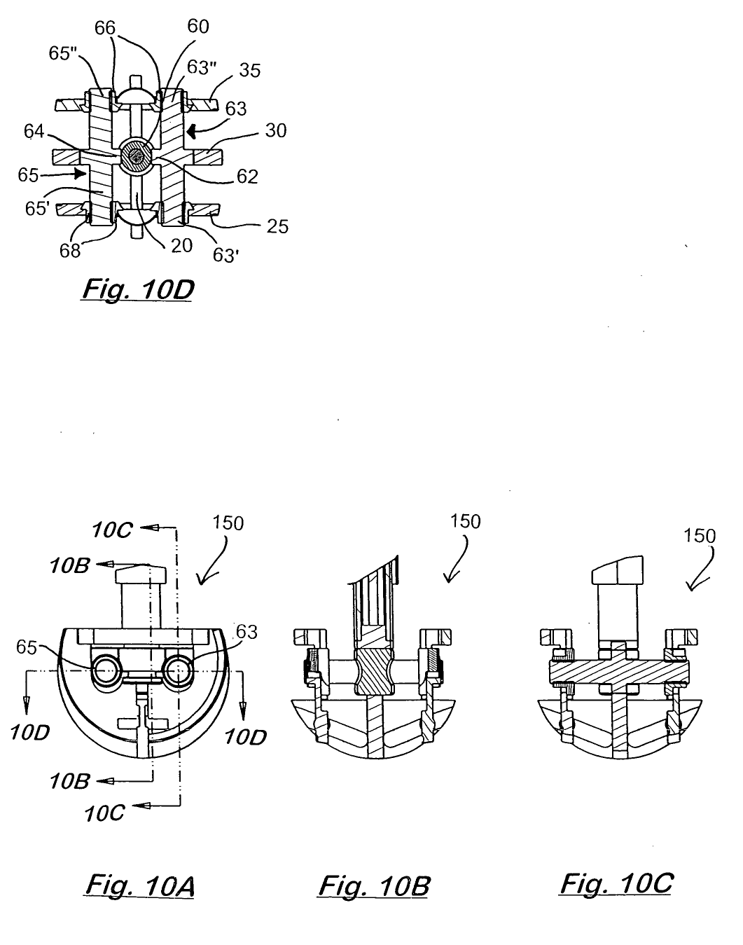

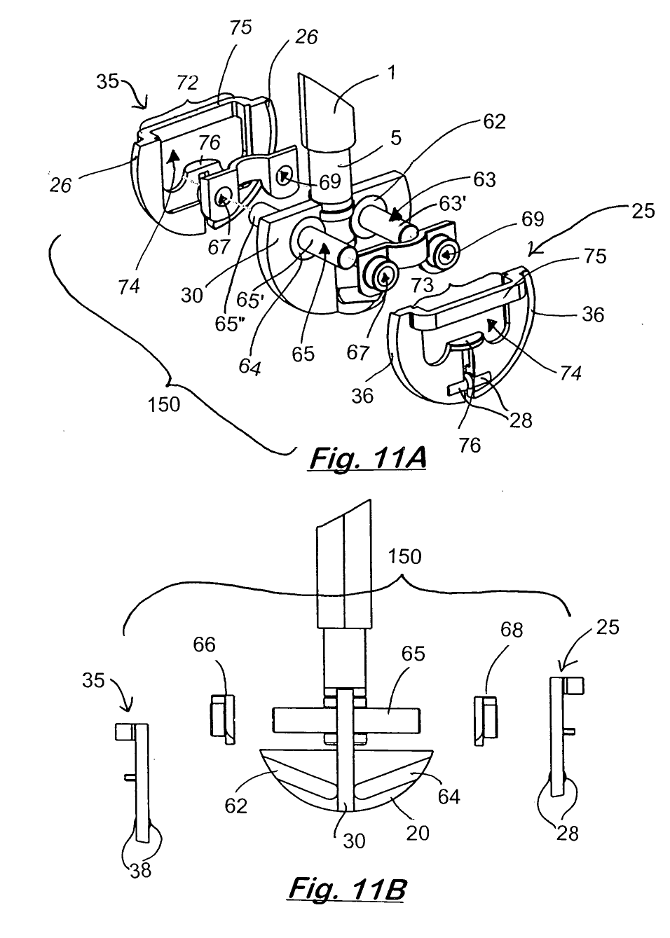

The expansion control rod or “center rod” 15 preferably extends down from the sun gear 50 through the shaft 5 and is coaxial and fixed with the center of a worm gear 60 (see FIGS. 5, 6A-6B, 10A-D, and 11A). Preferably, the worm gear 60 is right hand threaded to mesh with two center toothed portions 62, 64 on two cooperating worms 63, 65. The two center portions 62, 64 of the worms 63, 65 are rotatably mounted in, or otherwise extending through, the central blade 30. Preferably, worm 63 has left hand threads on one of its ends 63′ and right hand threads 63″ on the other of its ends (FIGS. 10D and 11A). The threads on the two ends of worm 65 are oriented to be opposite those of worm 63, so that end 65′ of worm 65 has right hand threads, and end 65″ of worm 65 has left hand threads.

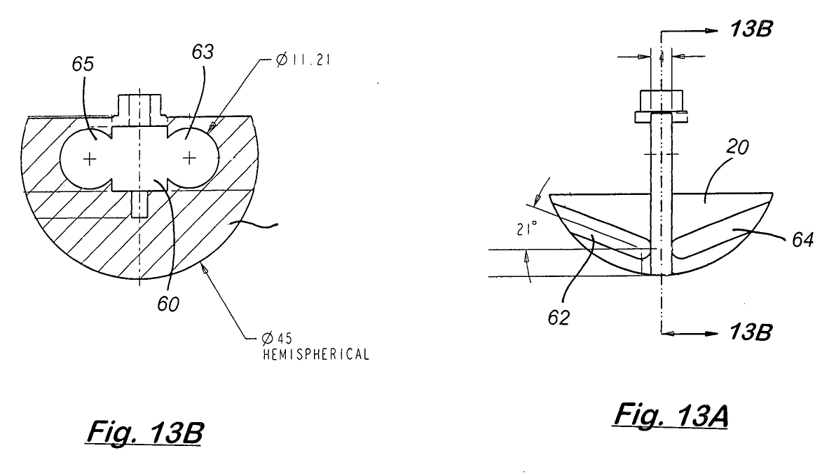

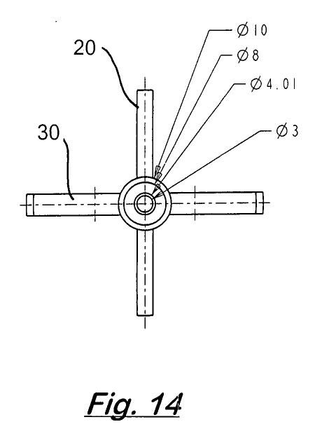

FIGS. 13A-13B and FIG. 14 illustrate the relationship between the guide blade 20 and the central blade 30. The central blade 30 and the guide blade 20 are perpendicular to one another, as shown in FIG. 14. One or both of them may have sharp edges, so that they are adapted to aid in cutting, and one or both cut/ream bottom B of the acetabulum, as shown in FIGS. 18A and 18B. One or both of blades 20, 30 is/are useful in moving debris (i.e. cut bone or other material) out of the way. Preferably, the radius of curvature for both the guide blade 20 and central blade 30 is designed to be the radius of the smallest hemisphere that is to be cut in the acetabulum; in this case the smallest radius of curvature is 45 mm, however other preferred sizes may be used. As shown in FIGS. 11B and 13B, the guide blade 20 comprises channels 22, 24; for the preferred head 150 that expands from a diameter of 45 to 53 mm, the channels are at a 21 degree slope, that is, at an angle 21 degrees from a plane that is perpendicular to the axis of rotation.

As shown in FIGS. 11A and 11B, two gear plates 66, 68 threadably engage over the ends of the worms 63, 65. The gear plates 66, 68 comprise threaded cylinders 67, 69 for receiving the ends of the worms 63, 65. Preferably, the gear plates 66, 68 comprise two slightly flared edges 70, 71 for being inserted into a mortise 72, 73 (see FIG. 11A) on the cutting blades 25, 35 similar to the connection used in a dovetail joint. Other slidable connection means may be used to capture the gear plates 66, 68 in the cutting blades 25, 35, or the cutting blades 25, 35 in the gear plates 66, 68, so that as the blades 25, 35 move with the gear plates 66, 68 along the worms 63, 65.

The gear plates 66, 68 preferably move out along the worm ends 63′, 63″ and 65′, 65″ due to the rotation of the worms 63 and 65 and the threaded engagement of cylinders 67 and 69 and worm threads. The preferred worm gear 60 is a 3.58 degree, right-hand, one-lead worm gear, with toothed surface 161, but other worm gears 60 and cooperating worms 63, 65 could be used. When the knob 40 is turned clockwise (as viewed in FIGS. 3 and 7), the ring 46 and planet gears 47, 48, and 49 turn clockwise, and the sun gear 50 and rod 15 turn counterclockwise. Therefore, worm gear 60 turns counterclockwise (see FIGS. 3 and 11A), worm 63 rotates counterclockwise (FIG. 10A) and worm 65 rotates clockwise (FIG. 10A). Thus, the worm gear 60 rotates the worms 63, 65 in opposite directions and the threaded ends 63′, 63″ and 65′, 65″ of the worms push both blades 25, 35 outward. The preferred planetary transmission and worm gear system allows the reverse actions to be done, that is, turning the knob 40 counterclockwise, which results in the worm gear 60 rotating clockwise (FIGS. 3 and 11A), and worms 63, 65 rotating clockwise and counterclockwise, respectively (FIG. 10A), to retract the gear plates 66, 68 and blades 25, 35 in toward he central axis of the device. Likewise, the indicator disk 52 will move in the opposite direction to indicate the retraction of the blades.

The slidable connection between the mortises 72, 73 and edges 70, 71 and the apertures 74, 75 allow the blades 25, 35 to slide up relative to the gear plates 66, 68 (guided by the channels 22, 24 in the guide blade 20) as the gear plates carrying the blades are moved outward. Both cutting blades 25, 35 comprise two braces 75, 76 for strength and rigidity. Preferably, the leading edges 26, 36 of each side of the cutting blades 25, 35 are sharp to enable cutting of the acetabulum the entire time the reamer is rotating.

Referring specifically to FIGS. 16A-18B:

In use, the preferred embodiment is utilized in a hip arthroplasty. After the incision is made along the patient's hip joint, the hip joint is exposed and the femoral head is resected. This allows visualization of the acetabulum. The acetabulum is then cleared of debris and the reamer 100 is then fixed to a surgical drill and inserted in the acetabular space in order to enlarge the acetabulum. The surgeon holds the drill in one hand and the reamer 100, preferably by the handle sleeve 1, in the other hand as he drills into the acetabulum. As the leading edges 26, 36 of the cutting blades 25, 35 spin due to the rotation of the reamer head 150 by the reamer shaft 5, they cut a first, small hemisphere in the acetabulum. Once the reamer 100 has made the first hemisphere it cannot cut a larger hemisphere until it is expanded. Therefore, cutting is stopped momentarily and the surgeon rotates the knob 40 which in turn expands the cutting blades 25, 35 to cut the next larger hemisphere of a size chosen by the surgeon. The surgeon continues to expand the blades 25, 35, removing subcondral bone until he has reached cancellous bone, which will grow into the prosthetic socket, and has reached the desired acetabular shape.

As illustrated in FIGS. 16A-16E the preferred acetabular reamer can expand from 45 mm to 53 mm cutting diameter: FIG. 16A shows the reamer expanded to 45 mm, FIG. 16B shows the reamer expanded to 47 mm, FIG. 16C shows the reamer expanded to 49 mm, FIG. 16D shows the reamer expanded to 51 mm, and FIG. 16E shows the reamer expanded to 53 mm. While incremental expansions are shown in FIGS. 16A-16E, the expansion of the preferred embodiment is continuous rather than incremental. The inventor envisions that another reamer size will be made that expands continuously within the range of 54 mm-64 mm. The 45 mm-53 mm size reamer will work for about 80% of the patients, and the 54 mm-64 mm will accommodate the other 20%. Other reamer sizes may be manufactured as well. The expansion of the cutting blades 25, 35 may be adjusted without needing to remove the reamer head from the acetabulum. Reaming devices according to the invention may be made with gearing or other blade adjustment systems that adjust the blades continuously, incrementally, and/or even automatically.

As the blades 25, 35 expand, they are also raised along the channels 22, 24 in the guide plate by means of slide protrusions 28, 38 in the cutting blades 25, 35; the protrusions 28, 38 slide in the channels 22, 24. This properly expands the effective diameter of the reamer head while maintaining a proper cutting curvature in the lower region of the reamer head. In other words, during the expansion and rising of the blades 25, 35, they substantially follow the radius of the central blade 30 and guide blade 20 in order to maintain nearly a perfectly hemispherical shape. If the blades 25, 35 were not raised at the same time they are expanded, they would not truly be spherical segments of an effectively-spherical reamer head, and rotation of the reamer head would result in there being a raised, non-reamed ring on the otherwise generally concave surface being reamed, in the location just inward from the inner surfaces of the blades 25, 35. Such an incongruity would not be acceptable for hip arthroplasty, for example.

If the preferred embodiment, central blade 30 and transverse guide blade 20 are designed to define the radius of the fully-contracted reamer head, with the cutting blades 25, 35 also defining the full-contracted reamer head radius in that they are slightly smaller in radius but also slightly distanced from the central axis of the reamer head. See FIG. 16A and schematic 17A. When the cutting blades 25, 35 begin to move out and up, they define the radius of the expanding reamer head as they become the “cutting segments” discussed earlier in this Description (see FIGS. 16B-E and also FIG. 17B). The central blade 30 and/or guide blade 20 continued to define the cutting radius of the bottom region of the reamer head (cutting/reaming bottom B region of the reamed surface), and so, because they exhibit the fully-contracted reamer head radius, there will be a very slight difference between the radius of the side S cutting edge(s) and the bottom B cutting edge(s). This difference is so small, especially until the reamer head is fully-expanded, that the reamer head effectively maintains nearly a perfect cutting sphere. A hemisphere cut by the preferred reamer is only 0.2-0.3 mm from having an absolutely perfect radius, and that is only if the reamer 100 is fully expanded (see FIG. 16E).

Due to the practical constraints of desiring a rotating shaft or other power source, and preferably a handle, connected to the reamer head, the term “cutting sphere” herein is used even through, in most embodiments, the cutting segments constructed as sharpened plates or other sharpened blades will tend not to be complete circles. Therefore the “cutting sphere” will typically, in effect, have a “spherical cap” removed or absent and the “cutting segment” will typically, in effect, have a “segment of a circle” removed or absent, to give room for the shaft, power source, handle and/or other structure. Therefore, the terms “cutting sphere,” “circular,” “spherical” and “spherical segment” herein do not necessarily require the object extend 360 degrees to be exactly a complete sphere, complete segment of a complete sphere, or a complete circle.

The preferred reamer may be expanded to the extent that the slide protrusions 28, 38 reach the end of the channels 22, 24 and exit the channels 22, 24, and then the cutting blades 25, 35 will “fall-off” the gear plates 66, 68. This feature is to allow easy removal of the blades for easy cleaning. If the surgeon continues to rotate the knob 40 after the cutting blades 25, 35 have been removed, the gear plates 66, 68 will also “fall-off” the worms in order to be cleaned. While reaming the acetabulum, the surgeon will stop expanding before the point at which the cutting blades or gear plates fall off the reamer head. Alternatively, there may be stops on the ends of the worms 63, 65, or other retaining structure, to prevent the blades 25, 35 and gear plates 66, 68 from “falling off”. The stops or other retaining structure would preferably be easily removable, to allow easy disassembly for cleaning and autoclaving, or for blade replacement or maintenance.

The reamer 100 reduces potential surgical injury to the soft tissue around the joint (sciatic nerve, vessels, and muscle), as well as being more efficient. As shown in FIGS. 18A and 18B, the reamer 100 is manufactured to be greater than 180 degrees in order to allow the reamer to cut a hemisphere even if the reamer 100 is not aligned with the axis of the acetabulum. The reamer 100 is preferably made of titanium, however other materials may be used, such as surgical steel.

While the above description focuses on expansion of the preferred reamer head, it is to be understood, and is understandable from the description and drawings, that the preferred knob and gearing system may be turned in the opposite direction to contract the size of the reamer head. During contraction of the reamer head 150, the knob 40 may be turned in the opposite direction as for expansion, the various gears will also turn in the opposite direction, and the cutting blades 25, 35 will ride on the plates 66, 68 inward toward the central axis of the device.

Cutting segments that are moveable outward and upward on the cutting/reaming head provide a head that is capable of more perfectly-spherical or perfectly-part-spherical cutting/reaming compared to other expandable reamers of which the inventor is aware. For example, an expandable reamer that has cutting blades that pivot outward and down from pivot points near the bottom of the reamer head will tend to produce incongruities and/or inaccuracies in the reamed surface. Such a pivoting-blade reamer head may be designed to cut a fairly accurate hemisphere at only one configuration, for example, either when fully-contracted, or when fully-expanded, but not both.

While the preferred reaming device is especially useful for hip arthroplasty, the device may have other uses, and embodiments may be adapted for the special requirements of other uses.

In view of the above summary and detailed description, some embodiments of the reaming device may be described as comprising a reamer head; and a shaft operatively connected to the reamer head for rotating the reamer head on a reamer head axis; wherein the reamer head comprises a moveable first blade having an outer edge on a first plane that is parallel to the reamer head axis, wherein the outer edge curves preferably greater than 180 degrees on a first radius and the outer edge has at least a sharpened portion; wherein the first blade is moveable in a direction perpendicular to said first plane out away from said reamer head axis, so that the effective cutting diameter of the rotating head is increased. While it is certainly preferred that there are multiple moveable blades, to better balance the reamer head and increase total cutting edge, the broad invention includes even a single one of said moveable blades. In some embodiments, the outer edge of said first blade preferably curves between 200 and 270 degrees on said plane, but may curve different amounts. The first blade preferably is a generally circular plate.

In other embodiments, the reamer head may further comprise a moveable second blade having an outer edge on a second plane that is parallel to the reamer head axis on a side of the reamer head axis opposite from said first blade, wherein the second blade outer edge curves preferably greater than 180 degrees on a second radius and has at least a sharpened portion; and wherein said second blade is moveable in a direction perpendicular to said second plane out away from said reamer head axis. Preferably, said first radius and said second radius are equal in length. Preferably, the outer edge of the first blade curves between 200-270 degrees on said first plane and the outer edge of said second blade curves between 200-270 degrees on said second plane. Preferably, the sharpened portion of the first blade outer edge extends substantially the entire length of the outer edge, and the sharpened portion of the second blade outer edge extends substantially the entire length of the outer edge, but other lengths of portions and/or multiple portions on a blade may be used.

Preferably, the reamer head is configured to move said first blade and second blade in a direction parallel to the reamer head axis at the same time the moveable blades move outward away from said reamer head axis. The drawings and above description illustrate sloped channels as one means of accomplishing this movement diagonal to the reamer head axis, but other means may be used to index the blades to move up at the same time as moving outward.

The reamer head may comprises a transverse blade perpendicular to the first blade and having an outer perimeter curving on a transverse blade radius, wherein the reamer head is configured so that, when the first blade moves outward away from said reamer head axis and also moves parallel to the reamer head axis, a bottom edge portion of the outer edge of the first blade stays aligned with the outer perimeter of the transverse blade. Likewise, the transverse blade may be perpendicular to a second blade, so that, when the first blade and the second blade each move outward away from said reamer head axis and also move parallel to the reamer head axis, a bottom edge portion of the first blade and a bottom edge of the second blade each stay aligned with the outer perimeter of the transverse blade. Said transverse blade may extend through the reamer head axis and said outer perimeter may have a sharpened bottom portion configured to ream a bottom surface generally perpendicular to the reamer head axis. The reamer head may comprise a central blade extending through the reamer head axis and having a bottom sharpened edge configured to ream a surface generally perpendicular to the reamer head axis.

In other embodiments, the device may be described as being for forming a concave surface, the device having a cutting head rotatable on a head axis, the cutting head having a first and second blade on opposite sides of the head axis, the first and second blades being moveable outward from the head axis from a contracted position to a expanded position, wherein said first and second blade are parallel to each other and to the head axis in both the contracted position and the expanded position. The head further may comprise a third blade parallel to and extending through the head axis and having a sharpened bottom perimeter edge, wherein said first and second blades are each generally circular and each has a sharpened circumferential edge, so that, when the head is rotated on the head axis, with the first and second blades in the contracted position, the sharpened circumferential edges together with the bottom perimeter edge define a cutting sphere having a first diameter, and when the head is rotated on the head axis, with the first and second blades in the expanded position, the sharpened circumferential edges together with the bottom perimeter edge define a cutting sphere having a second diameter larger than said first diameter. Said first and second blades may have equal diameters. Said first and second blades may be configured to move upward parallel to the head axis when moving from the contracted position to the expanded position, so that said first and second blades are higher on said head in the expanded position than in the contracted position.

The devices may further comprise a shaft connected to said head coaxial with said head axis and a surgical drill operatively connected to the shaft for rotating the head to ream a bone surface.

Although this invention has been described above with reference to particular means, materials and embodiments, it is to be understood that the invention is not limited to these disclosed particulars, but extends instead to all equivalents within the scope of the following claims.

I claim:

1. A reaming device comprising:
a reamer head;
a shaft operatively connected to the reamer head for rotating the reamer head on a reamer head axis;
wherein the reamer head comprises a moveable first blade having an outer edge on a first plane that is parallel to the reamer head axis, wherein the outer edge curves greater than 180 degrees on a first radius and the outer edge has a sharpened portion;
wherein the first blade is moveable in a direction perpendicular to said first plane out away from said reamer head axis, so that the effective cutting diameter of the rotating head is increased.

2. The reaming device of claim 1, wherein the outer edge of said first blade curves between 200 and 270 degrees on said plane.

3. The reaming device of claim 1, wherein the first blade is a generally circular plate.

4. The reaming device of claim 1, wherein the reamer head further comprises:
a moveable second blade having an outer edge on a second plane that is parallel to the reamer head axis on a side of the reamer head axis opposite from said first blade, wherein the second blade outer edge curves greater than 180 degrees on a second radius and has a sharpened portion;
wherein said second blade is moveable in a direction perpendicular to said second plane out away from said reamer head axis.

5. The reaming device of claim 4, wherein said first radius and said second radius are equal in length.

6. The reaming device of claim 4, wherein the outer edge of said first blade curves between 200-270 degrees on said first plane and the outer edge of said second blade curves between 200-270 degrees on said second plane.

7. The reaming device of claim 1, wherein the sharpened portion of the first blade outer edge extends substantially the entire length of the outer edge.

8. The reaming device of claim 4, wherein the sharpened portions of the outer edges of the first blade and second blade each extend substantially the entire length of their respective outer edges.

9. The reaming device of claim 1, wherein the reamer head is configured to move said first blade in a direction parallel to the reamer head axis at the same time the first blade moves outward in said direction perpendicular to said first plane out away from said reamer head axis.

10. The reaming device of claim 4, wherein the reamer head is configured to move said first blade and also said second blade in a direction parallel to the reamer head axis at the same time the first blade and said second blade move outward away from said reamer head axis.

11. The reaming device of claim 9, wherein the reamer head further comprises a transverse blade perpendicular to the first blade and having an outer perimeter curving on a transverse blade radius, wherein the reamer head is configured so that, when the first blade moves outward away from said reamer head axis and also moves parallel to the reamer head axis, a bottom edge portion of the outer edge of the first blade stays aligned with the outer perimeter of the transverse blade.

12. The reaming device of claim 10, wherein the reamer head further comprises a transverse blade perpendicular to the first blade and the second blade and having an outer perimeter curving on a transverse blade radius, wherein the reamer head is configured so that, when the first blade and the second blade each move outward away from said reamer head axis and also move parallel to the reamer head axis, a bottom edge portion of the first blade and a bottom edge of the second blade each stay aligned with the outer perimeter of the transverse blade.

13. The reaming device of claim 11, wherein said transverse blade extends through the reamer head axis and said outer perimeter has a sharpened bottom portion configured to ream a surface generally perpendicular to the reamer head axis.

14. The reaming device of claim 12, wherein said transverse blade extends through the reamer head axis and said outer perimeter has a sharpened bottom portion configured to ream a surface generally perpendicular to the reamer head axis.

15. The reaming device of claim 1, wherein the reamer head further comprises a central blade extending through the reamer head axis and having a bottom sharpened edge configured to ream a surface generally perpendicular to the reamer head axis.

16. The reaming device of claim 4, wherein the reamer head further comprises a central blade extending through the reamer head axis and having a bottom sharpened edge configured to ream a surface generally perpendicular to the reamer head axis.

17. The reaming device of claim 1, further comprising a surgical drill connected to said shaft for rotating the reamer head on a reamer head axis for reaming a bone surface.

18. A device for forming a concave surface, the device having a cutting head rotatable on a head axis, the cutting head having a first and second blade on opposite sides of the head axis, the first and second blades being moveable outward from the head axis from a contracted position to a expanded position, wherein said first and second blade are parallel to each other and to the head axis in both the contracted position and the expanded position.

19. The device of claim 18, wherein the head further comprises a third blade parallel to and extending through the head axis and having a sharpened bottom perimeter edge, wherein said first and second blades are each generally circular and each has a sharpened circumferential edge, so that, when the head is rotated on the head axis, with the first and second blades in the contracted position, the sharpened circumferential edges together with the bottom perimeter edge define a cutting sphere having a first diameter, and when the head is rotated on the head axis, with the first and second blades in the expanded position, the sharpened circumferential edges together with the bottom perimeter edge define a cutting sphere having a second diameter larger than said first diameter.

20. The device of claim 19, wherein said first and second blades have equal diameters.

21. The device of claim 19, wherein said first and second blades are configured to move upward parallel to the head axis when moving from the contracted position to the expanded position, so that said first and second blades are higher on said head in the expanded position than in the contracted position.

22. The device of claim 18, further comprising a shaft connected to said head coaxial with said head axis and a surgical drill operatively connected to the shaft for rotating the head to ream a bone surface.
